(19) United States
(12) Patent Application Publication
(10) Pub. No.: US 2020/0271574 A1
(43) Pub. Date: Aug. 27, 2020

(54) INFRARED DETECTOR ASSEMBLY WITH INTEGRATED TEMPERATURE SENSING, GAS MEASUREMENT APPARATUS METHOD

(71) Applicant: KONINKLIJKE PHILIPS N.V., EINDHOVEN (NL)

(72) Inventors: DAVID SCAMPOLI, SOUTH GLASTONBURY, CT (US); CHARLES CHACKO, ORANGE, CT (US)

(73) Assignee: KONINKLIJKE PHILIPS N.V., EINDHOVEN (NL)

(21) Appl. No.: 16/648,733

(22) PCT Filed: Sep. 21, 2018
(86) PCT No.: PCT/EP2018/075550

§ 371 Date: Mar. 19, 2020

Related U.S. Application Data
(60) Provisional application No. 62/566,576, filed on Oct. 2, 2017.

(51) Int. Cl. G01N 21/3504; A61B 5/083; G01J 5/06; G01J 5/20

(52) U.S. Cl. CPC G01N 21/3504; A61B 5/0836; A61B 5/082; G01J 5/20; G01J 5/06

(57) ABSTRACT

A method of making an infrared detector assembly (10) with integrated temperature sensing comprises forming at least one IR sensitive element (12,14) on a substrate (16) and forming conductive electrode pads (22,24,26,28,30,32) for (a) IR sensitive element and (b) at least one thermistor (34) on the substrate. The conductive electrode pads and the IR sensitive element are in a centerline symmetrical configuration in which the conductive electrode pads and the IR sensitive element, taken together, are centerline symmetrical about at least one axis (36,38) in a plane of the infrared detector assembly, wherein the centerline symmetrical configuration is operable to reduce a thermal lag time between a temperature of the at least one thermistor and a temperature of the IR sensitive element during temperature transients. Each of first and second thermistor conductive electrode pads (30,32) has two pad end portions (40,42) spaced from each other and joined via a pad mid-portion (44) that comprises a thermal loss reduction member.

The present embodiments relate generally to infrared detectors and more particularly, to an infrared detector with integrated temperature sensing, gas measurement apparatus with the same, a method of measuring a gas concentration, and a method of making the same.

Lead selenide detectors, used to detect to mid-range infrared energy, are sensitive to changes in ambient temperature. As the ambient temperature of the detector drifts, so does the responsivity of the detector. As a result, applications that use lead selenide detectors must be thermally regulated, or mathematically compensated for the drift in sensitivity. Methods to do this require close thermal coupling with the substrate of the lead selenide detector, and is typically done with a thermistor mounted externally to the lead selenide detector. However, such a method disadvantageously infers the temperature of the lead selenide based on a measurement external to the detector, and does not directly measure the temperature of the detector itself.

Many capnography systems use two IR detectors, such as lead selenide detectors. One detector is for detecting a sample gas absorption wavelength and the other detector senses a reference wavelength. Both detectors must be closely monitored for any small changes in temperature between the two detectors. One example of such a capnography system is described in co-assigned U.S. Patent Publication No. 2013/0292570 entitled “System and method for performing heater-less lead selenide-based capnometry and/or capnography”, which is herein incorporated by reference.

Prior art capnography systems such as the example provided also sense temperature at the detector by placing a thermistor in proximity to the lead selenide plate detector, typically mounted onto a substrate surface, with an intermediate layer in-between, and upon which the detector body is also mounted. Unfortunately, this arrangement introduces a large thermal gradient and an associated large thermal lag time between the thermistor sensor and the film temperature of the lead selenide plate detector. Such a large thermal gradient and large thermal lag time occur because the substrate on which the film is deposited is made of fused quartz, a poor thermal conductor, and because of the poor thermal conductivity of the intermediate layer or layers.

Accordingly, what is needed is a more accurate and faster response detection of the lead selenide plate detector, especially as to the effects of temperature variation, which avoids the problems presented by the prior art. An improved method and apparatus for overcoming the problems in the art is desired.

In accordance with one aspect, an apparatus and method are disclosed which advantageously overcomes the problems in the art by integrating a thermistor directly on the substrate of a lead selenide (PbSe) detector assembly, at the closest possible point to the lead selenide detector element. As a result, the temperature of the lead selenide detector element can be more accurately measured. In addition, temperature compensation by mathematical algorithm or thermal regulation can be more precise, thus advantageously eliminating detector drift due to changes in detector temperature.

In accordance with another aspect, an infrared detector includes the addition of at least one of (i) a thermistor or (ii) thermistor chemistry directly on the same substrate as the lead selenide detector element, with either a single detector element or a plurality of detector elements being monitored by a single or a plurality of thermistor elements.

The embodiments of the present disclosure advantageously solve the problem of changes in the temperature of the lead selenide which can now be more accurately measured. In addition, temperature compensation by mathematical algorithm or thermal regulation can be more precise, thus advantageously eliminating detector drift due to changes in detector element temperature.

The embodiments of the present disclosure have utility, in particular, for carbon dioxide gas detection and measurement, as well as for detection and measurement of any other gas with absorption wavelengths in the mid-wave infrared spectral band. The inventors have discovered an ingenious and novel arrangement of conductive electrode pads for a temperature sensor with respect to an IR-sensitive detector film. Such a temperature sensor can comprise a micro-miniature chip thermistor or a resistive thermistor chemistry deposition. In addition, as will become better understood from the disclosure herein, with a chip thermistor mounted, or resistive thermistor chemistry deposition, immediately adjacent to each of the two IR-detectors, and separated there from by a thermal coupling separation spacing, any small differential temperatures between the two detectors can then be detected and algorithmically compensated for to maintain the capnography system CO₂ accuracy. The method according to the embodiments of the present disclosure could be used on any other detector material or assemblies in which electrical conductive pad terminals exist in close proximity to the detector sensing material.

In one embodiment, the inventive approach to increasing the temperature measurement and temperature tracking accuracy of a detector such as the lead selenide (PbSe) plate detector also involves mounting a micro-miniature chip thermistor onto the gold plated electrode immediately adjacent to the lead selenide detector without coming into contact with the lead selenide film. The chip thermistor may be surface mounted onto two gold plated electrodes, which are deposited concurrently with gold plated electrodes for the IR-sensitive film, but subsequent to deposition of the IR-sensitive film, onto a fused quartz substrate. Such an arrangement arose from the realization that the electrode ends of the detector film are both electrically and thermally conductive. The mounting of the temperature sensor, e.g., thermistor, immediately adjacent to the lead selenide film, places the temperature sensor at a position with respect to the lead selenide film for minimizing thermal conductivity losses, and minimizing thermal lag time, with respect to the film, and at the same time offering an electrical junction for sensor electrical communication. Reduced cost of circuitry and reduced space requirements are also advantageously realized.

According to one embodiment, a method of making an infrared detector assembly with integrated temperature sensing comprises forming at least one infrared radiation sensitive element or IR sensitive element directly on a substrate, wherein the at least one IR sensitive element is thermally coupled to the substrate. The method further includes forming conductive electrode pads for (a) the at least one IR sensitive element and (b) at least one thermistor directly on the substrate, wherein the conductive electrode pads are thermally coupled to the substrate. The conductive electrode pads and the at least one IR sensitive element are in a centerline symmetrical configuration in which the conductive electrode pads and the at least one IR sensitive element, taken together, are centerline symmetrical about at least one axis in a plane of the infrared detector assembly. The centerline symmetrical configuration is operable to reduce a thermal lag time between a temperature of the at least one thermistor and a temperature of the at least one IR sensitive element during temperature transients of the infrared detector assembly.

Forming the conductive electrode pads comprises depositing and patterning a conductive material overlying the substrate into (i) at least one pair of first and second IR sensitive element conductive electrode pads directly on the substrate for each of the at least one IR sensitive element and (ii) first and second thermistor conductive electrode pads directly on the substrate for each of the at least one thermistor. Each pair of first and second IR sensitive element conductive electrode pads electrically couple to a respective at least one IR sensitive element via an edge portion of the respective at least one IR sensitive element overlapped by an edge portion of each respective pad of the pair of first and second IR sensitive element conductive electrode pads. Each of the first and second thermistor conductive electrode pads has a plan view geometry of two pad end portions spaced along a length dimension of a respective thermistor conductive electrode pad, the two pad end portions having length and width dimensions and being joined via a pad mid-portion. The pad mid-portion comprises a thermal heat loss reduction member having a width dimension less than its length dimension. The width dimension of the pad mid-portion is less than the respective width dimension of each of the two pad end portions. In addition, each of the first and second thermistor conductive electrode pads extend in tandem along a line parallel to the length dimension of the at least one IR sensitive element, immediately adjacent to the at least one IR sensitive element and separated there from by a thermal coupling separation spacing.

The method further comprises performing one selected from the group consisting of (i) forming the at least one thermistor directly on the substrate via a deposited resistive thermistor chemistry and dicing the substrate with the conductive electrode pads, the at least one IR sensitive element, and the at least one thermistor into at least one individual infrared detector assembly and (ii) dicing the substrate with the conductive electrode pads and the at least one IR sensitive element into at least one individual partial infrared detector assembly, and completing the at least one individual partial infrared detector assembly by disposing the at least one thermistor directly on an individual diced substrate, via a surface mountable resistive thermistor chip.

In connection with forming the at least one thermistor directly on the substrate via the deposited resistive thermistor chemistry, each respective at least one thermistor is (a) thermally coupled to the substrate and (b) electrically coupled between opposite pad end portions nearest one another of a respective pair of the first and second thermistor conductive electrode pads. The opposite pad end portions nearest one another of the respective pair of the first and second thermistor conductive electrode pads are spaced from one another by a thermistor element deposition placement distance of the at least one thermistor.

In connection with dicing the substrate with the conductive electrode pads and the at least one IR sensitive element into at least one individual partial infrared detector assembly and disposing the at least one thermistor directly on an individual diced substrate, each respective at least one thermistor is (a) thermally coupled to the individual diced substrate and (b) electrically coupled between opposite pad end portions nearest one another of a respective pair of the first and second thermistor conductive electrode pads. The opposite pad end portions nearest one another of the respective pair of the first and second thermistor conductive electrode pads are spaced from one another by a surface mount thermistor placement distance of the at least one thermistor.

In accordance with another embodiment, the method includes wherein the substrate comprises a quartz substrate having a thickness in a range of 0.50 to 0.70 mm, and wherein the at least one IR sensitive element comprises a lead selenide film element. In addition, the thermal coupling separation spacing is in a range of 0.10 to 0.30 mm.

In another embodiment, the method further includes wherein (i) the at least one pair of first and second IR sensitive element conductive electrode pads and (ii) the first and second thermistor conductive electrode pads of the at least one thermistor comprise a single electrically conductive material or more than one electrically conductive material, wherein each of the more that one electrically conductive materials is of an at least 90-100% matched thermal conductivity. In a further embodiment, the conductive electrode pads comprise at least one of gold and platinum.

According to another embodiment, the method includes the centerline symmetrical configuration of the conductive electrode pads and the at least one IR sensitive element being operable to reduce the thermal lag time between a temperature of the at least one thermistor and a temperature of the at least one IR sensitive element to one second or less during temperature transients of the infrared detector assembly.

In a further embodiment, the at least one IR sensitive element comprises one selected from the group consisting of (i) a single IR sensitive element, (ii) two IR sensitive elements, and (iii) a plurality of IR sensitive elements. For instance, the at least one IR sensitive element can comprise two or more IR sensitive elements, wherein the conductive electrode pads and the two or more IR sensitive elements are in a centerline symmetrical configuration in which the conductive electrode pads and the two or more IR sensitive elements are centerline symmetrical about a first axis and a second axis, perpendicular to the first axis, in the plane of the infrared detector assembly.

In another embodiment, the at least one thermistor comprises two or more thermistors, wherein the conductive electrode pads and the at least one IR sensitive element are in a centerline symmetrical configuration in which the conductive electrode pads and the at least one IR sensitive element are centerline symmetrical about a first axis and a second axis, perpendicular to the first axis, in the plane of the infrared detector assembly. In still another embodiment, the at least one IR sensitive element comprises one or more IR sensitive elements, wherein the at least one thermistor comprises multiple thermistors, and wherein each of the at least one thermistors is disposed adjacent to at least one of the one or more IR sensitive elements.

According to another embodiment, an infrared detector assembly with integrated temperature sensing, comprises at least one infrared radiation sensitive element or IR sensitive element formed directly on a substrate, wherein the at least one IR sensitive element is thermally coupled to the substrate. The assembly further comprises conductive electrode pads formed directly on the substrate for (a) the at least one IR sensitive element and (b) at least one thermistor. The conductive electrode pads are thermally coupled to the substrate. The conductive electrode pads and the at least one IR sensitive element are in a centerline symmetrical configuration in which the conductive electrode pads and the at least one IR sensitive element, taken together, are centerline symmetrical about at least one axis in a plane of the infrared detector assembly. The centerline symmetrical configuration is operable to reduce a thermal lag time between a temperature of the at least one thermistor and a temperature of the at least one IR sensitive element during temperature transients of the infrared detector assembly.

The conductive electrode pads comprise (i) at least one pair of first and second IR sensitive element conductive electrode pads directly on the substrate for each of the at least one IR sensitive element, wherein each pair of first and second IR sensitive element conductive electrode pads electrically couple to a respective at least one IR sensitive element via an edge portion of the respective at least one IR sensitive element overlapped by an edge portion of each respective pad of the pair of first and second IR sensitive element conductive electrode pads. The conductive electrode pads further comprise (ii) first and second thermistor conductive electrode pads directly on the substrate for each of the at least one thermistor. Each of the first and second thermistor conductive electrode pads has a plan view geometry of two pad end portions spaced along a length dimension of a respective thermistor conductive electrode pad, the two pad end portions having length and width dimensions and being joined via a pad mid-portion. The pad mid-portion comprises a thermal heat loss reduction member having a width dimension less than its length dimension. The width dimension of the pad mid-portion is less than the respective width dimension of each of the two pad end portions. In addition, each of the first and second thermistor conductive electrode pads extend in tandem along a line parallel to the length dimension of the at least one IR sensitive element, immediately adjacent to the at least one IR sensitive element and separated there from by a thermal coupling separation spacing.

The infrared detector assembly with integrated temperature sensing further comprises at least one thermistor selected from the group consisting of (i) at least one thermistor formed directly on the substrate via a deposited resistive thermistor chemistry, and (ii) at least one thermistor mounted directly on the substrate that comprises a surface mountable resistive thermistor chip.

In connection with the deposited resistive thermistor chemistry, each respective at least one thermistor is (a) thermally coupled to the substrate and (b) electrically coupled between opposite pad end portions nearest one another of a respective pair of the first and second thermistor conductive electrode pads. In addition, the opposite pad end portions nearest one another of the respective pair of the first and second thermistor conductive electrode pads are spaced from one another by a thermistor element deposition placement distance of the at least one thermistor.

In connection with the surface mountable resistive thermistor chip, each respective at least one thermistor is (a) thermally coupled to the substrate and (b) electrically coupled between opposite pad end portions nearest one another of a respective pair of the first and second thermistor conductive electrode pads. In addition, the opposite pad end portions nearest one another of the respective pair of the first and second thermistor conductive electrode pads are spaced from one another by a surface mount thermistor placement distance of the at least one thermistor.

According to a further embodiment, a carbon dioxide gas measurement apparatus comprises an infrared detector assembly as discussed herein. The carbon dioxide gas measurement apparatus further comprises a circuit coupled to the infrared detector assembly and configured to (i) obtain a temperature measurement output from the at least one thermistor and (ii) provide a temperature compensated carbon dioxide gas measurement output signal based on the obtained temperature measurement, wherein the circuit compensates an output signal of the at least one IR sensitive element for a drift in temperature of the respective at least one IR sensitive element in response to the obtained temperature measurement.

In one embodiment, the at least one IR sensitive element of the carbon dioxide gas measurement apparatus comprises two IR sensitive elements. In this embodiment, the conductive electrode pads and the two IR sensitive elements are in a centerline symmetrical configuration in which the conductive electrode pads and the two IR sensitive elements are centerline symmetrical about a first axis and a second axis, perpendicular to the first axis, in the plane of the infrared detector assembly. In addition, one of the two IR sensitive elements is configured to output an IR reference signal, and the other of two IR sensitive elements is configured to output a carbon dioxide gas measurement signal, wherein both the reference signal and the carbon dioxide gas measurement signal are temperature compensated for a drift in temperature of each respective IR sensitive element in response to the obtained temperature measurement.

According to a still further embodiment, a method of measuring a gas concentration comprises: providing an infrared detector assembly as discussed herein; obtaining, via a circuit coupled to the infrared detector assembly, a temperature measurement output from the at least one thermistor; and providing, via the circuit, a temperature compensated carbon dioxide gas measurement output signal based on the obtained temperature measurement, wherein an output signal of the at least one IR sensitive element is compensated, via the circuit, for a drift in temperature of the respective at least one IR sensitive element in response to the obtained temperature measurement.

Still further advantages and benefits will become apparent to those of ordinary skill in the art upon reading and understanding the following detailed description.

The embodiments of the present disclosure may take form in various components and arrangements of components, and in various steps and arrangements of steps. Accordingly, the drawings are for purposes of illustrating the various embodiments and are not to be construed as limiting the embodiments. In the drawing figures, like reference numerals refer to like elements. In addition, it is to be noted that the figures may not be drawn to scale.

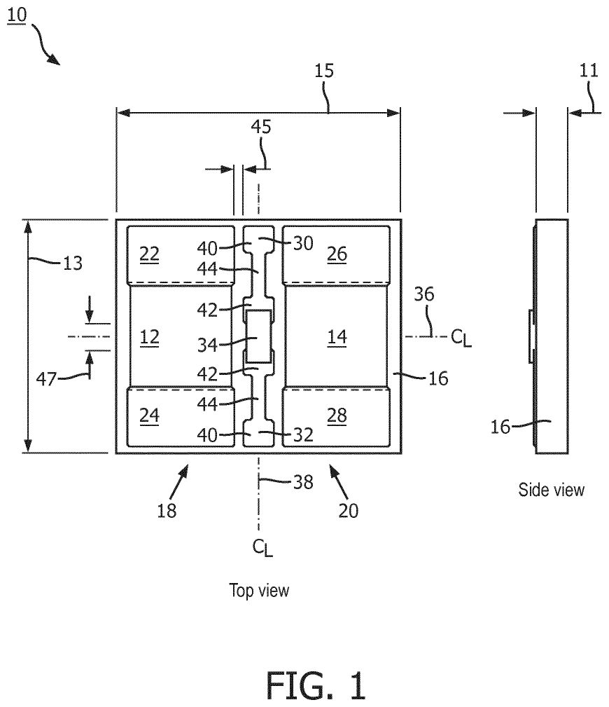

FIG. 1 is a top view and a side view of an IR detector assembly with a surface mount chip thermistor integrated with dual lead selenide detector elements, for use as reference and sample channels, on a substrate according to an embodiment of the present disclosure;

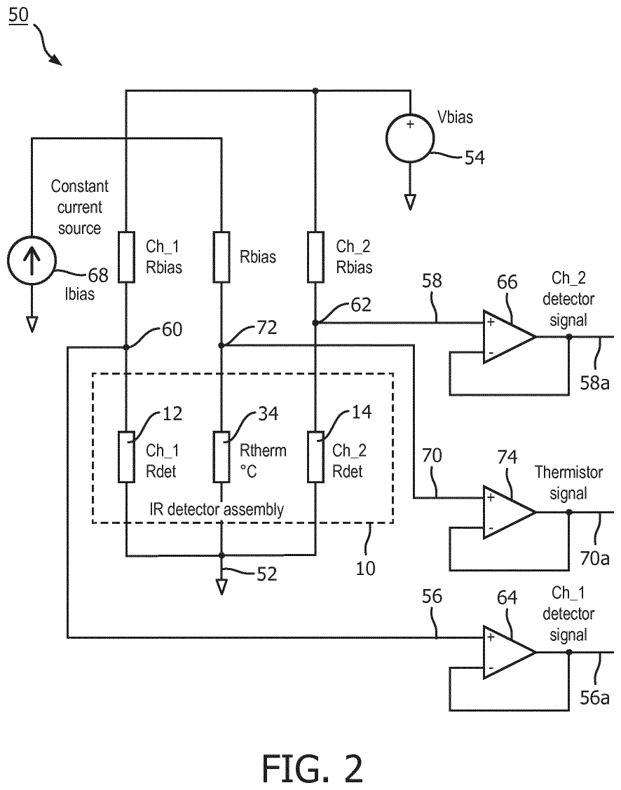

FIG. 2 is an electrical schematic of a detection circuit for use with a dual channel IR detector assembly having a single thermistor element (Rₜₕₑᵣₘ ° C.) and two lead selenide detector elements (Ch_1 R_{det} and Ch_2 R_{det}) according to an embodiment of the present disclosure;

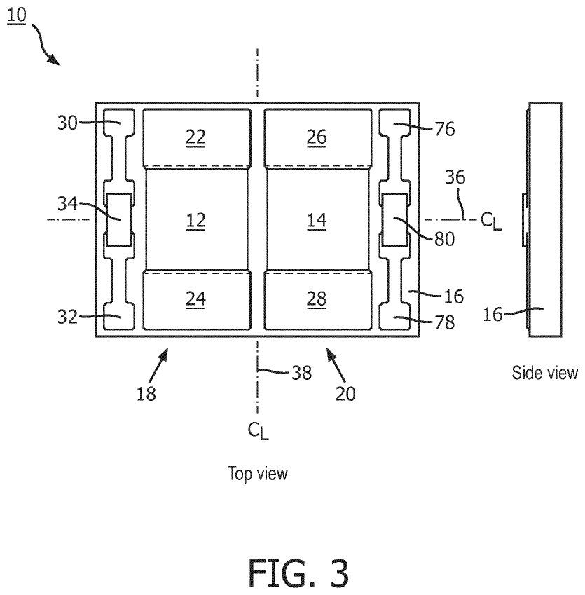

FIG. 3 is a top view and a side view of an IR detector assembly with dual, surface mount, chip thermistors integrated with dual lead selenide detector elements, for use as reference and sample channels, on a substrate according to an embodiment of the present disclosure;

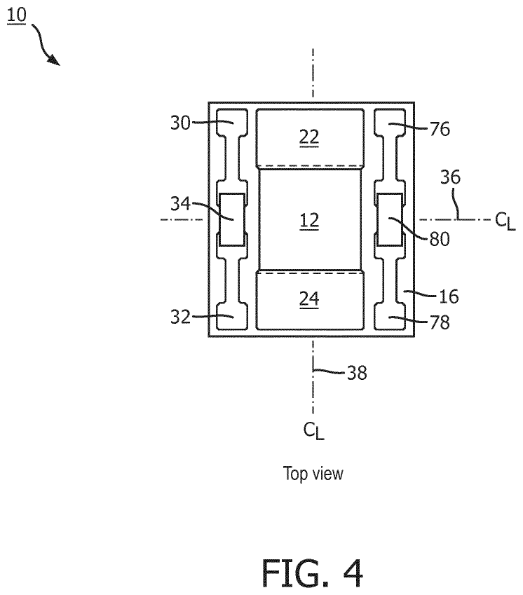

FIG. 4 is a top view of an IR detector assembly with dual, surface mount, chip thermistors integrated with a single lead selenide detector element, for use as one of a reference or a sample channel, on a substrate according to an embodiment of the present disclosure;

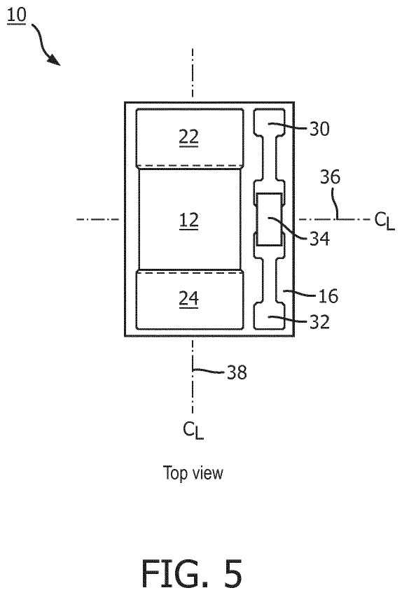

FIG. 5 is a top view of an IR detector assembly with a single surface mount chip thermistor integrated with a single lead selenide detector, for use as one of a reference or a sample channel, on a substrate according to an embodiment of the present disclosure;

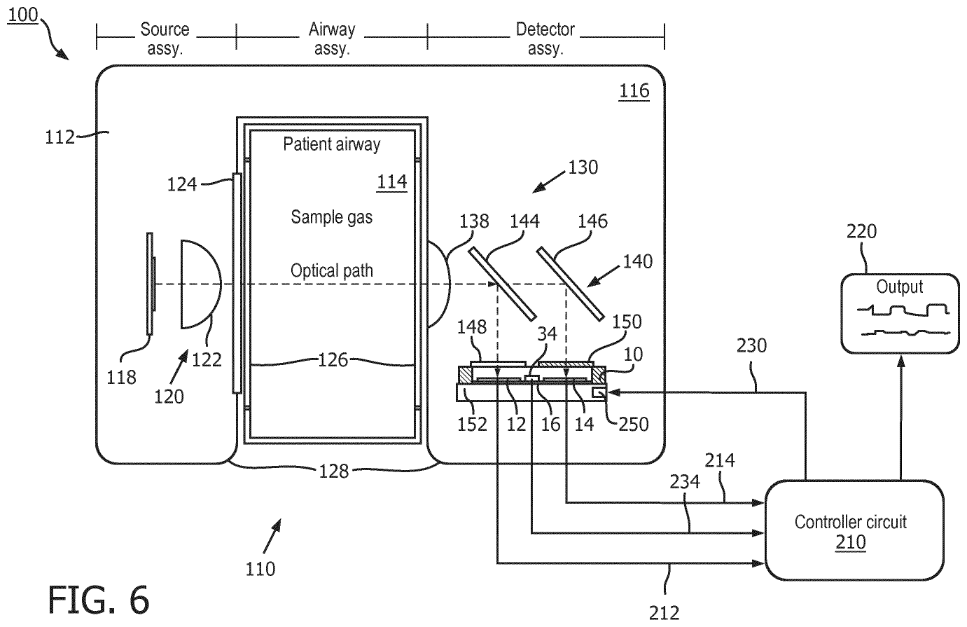

FIG. 6 illustrates an improved capnography system which includes an IR detector assembly with dual IR detector elements and an integrated temperature sensor according to an embodiment of the present disclosure; and

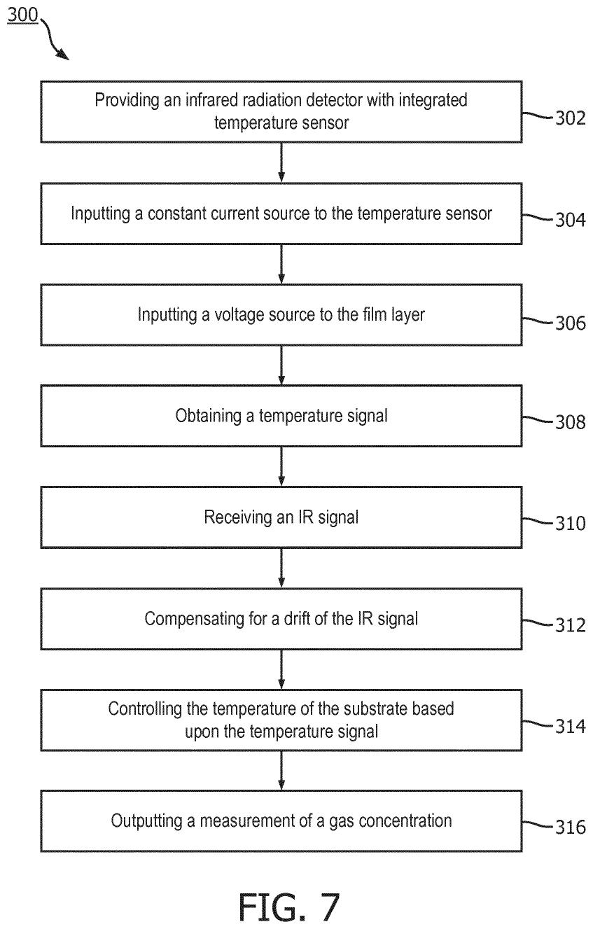

FIG. 7 is a flow chart illustrating a method of measuring a gas concentration according to another embodiment of the present disclosure.

The embodiments of the present disclosure and the various features and advantageous details thereof are explained more fully with reference to the non-limiting examples that are described and/or illustrated in the drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the present disclosure. The examples used herein are intended merely to facilitate an understanding of ways in which the embodiments of the present may be practiced and to further enable those of skill in the art to practice the same. Accordingly, the examples herein should not be construed as limiting the scope of the embodiments of the present disclosure, which is defined solely by the appended claims and applicable law.

It is understood that the embodiments of the present disclosure are not limited to the particular methodology, protocols, devices, apparatus, materials, applications, etc., described herein, as these may vary. It is also to be understood that the terminology used herein is used for the purpose of describing particular embodiments only, and is not intended to be limiting in scope of the embodiments as claimed. It must be noted that as used herein and in the appended claims, the singular forms “a,” “an,” and “the” include plural reference unless the context clearly dictates otherwise.

Unless defined otherwise, all technical and scientific terms used herein have the same meanings as commonly understood by one of ordinary skill in the art to which the embodiments of the present disclosure belong. Preferred methods, devices, and materials are described, although any methods and materials similar or equivalent to those described herein can be used in the practice or testing of the embodiments.

According to one embodiment, a quartz wafer is used as a substrate for the direct deposition and patterning of lead selenide detector elements, followed by deposition and patterning of gold conductive pad elements. The gold conductive pad elements provide a means to electrically connect to the lead selenide detector elements, in addition to one or more thermistor element. Lead selenide is sensitive to mid-range infrared radiation, and is used for mid-range IR micro spectroscopy of exhaled patient gases. By providing a specialized shape and geometry for the conductive electrode pads (e.g., gold conductive pads) and using a cutting pattern to separate the quartz wafer into lead selenide detector elements (e.g., a single detector, pairs of detectors, etc.), a location can be provided on an assembly to either (i) mount a commercially available thermistor, or (ii) deposit a resistive chemistry that changes resistance proportional to the temperature change. In one embodiment, a thermistor location is provided directly between two detector elements, giving a two-channel detector with a single-channel thermistor measurement centrally spaced between the two lead selenide detector elements, as will be discussed further herein. Other embodiments can have multiple detector elements with single or multiple thermistor elements for temperature monitoring, further as will be discussed herein.

With reference now to FIG. 1, a method of making an infrared detector assembly 10 with integrated temperature sensing comprises forming at least one infrared radiation sensitive element (indicated via reference numeral 12 or 14) or IR sensitive element directly on (e.g., without intervening layers) a substrate 16. Substrate 16 is of a suitable substrate material for forming electrical components, for example, a fused quartz material having a thickness, as indicated via reference numeral 11, in a range of 0.50 to 0.70 mm. In the illustration of FIG. 1, two IR sensitive elements 12 and 14 are formed, which provides for a dual channel detector assembly, including a first detector channel 18 and a second detector channel 20, as will be discussed further herein. The infrared detector assembly 10 of FIG. 1 is small in size, e.g., on the order of 4.6×5.6 mm, or smaller, as indicated via reference numerals 13 and 15, respectively. Other small size dimensions are also possible.

The at least one IR sensitive element (12, 14) is thermally coupled to the substrate 16. Each of the at least one IR sensisitve element (12, 14) can comprise a film layer of an infrared sensitive material that is disposed and patterned on the surface of substrate 16 by forming or adhering, using suitable techniques known in the art. A preferred IR sensitive material comprises lead selenide (PbSe). The lead selenide material is well known for having a resistivity that is a function of an amount of mid-range IR energy incident on the material, and thus is suitable for measuring IR radiation. The film layer is shaped to have two ends, across which is placed a voltage for measuring a desired characteristic, as will be discussed further herein.

The method further includes forming conductive electrode pads (indicated via reference numerals 22, 24, 26, 28, 30 and 32) for (a) the at least one IR sensitive element (12, 14) and (b) at least one thermistor 34 directly on the substrate 16 (e.g., without any intervening layers, and wherein the thermistor 34 is yet to be formed or mounted, as discussed further herein below). The conductive electrode pads (22, 24, 26, 28, 30 and 32) are thermally coupled to the substrate. The conductive electrode pads (22, 24, 26, 28, 30 and 32) and the at least one IR sensitive element (12, 14) are in a centerline symmetrical configuration in which the conductive electrode pads and the at least one IR sensitive element, taken together, are centerline symmetrical about at least one axis (indicated via reference numeral 36 or 38) in a plane of the infrared detector assembly 10.

In one embodiment, the at least one pair of first and second IR sensitive element conductive electrode pads ((22,24),(26,28)) and the first and second thermistor conductive electrode pads (30, 32) of the at least one thermistor 34 comprise a single electrically and thermally conductive material. In another embodiment, the electrode pads comprise more than one type of electrically and thermally conductive material, wherein each of the more that one type of electrically and thermally conductive materials is of an at least 90-100% matched thermal conductivity. For example, the conductive electrode pads can comprise at least one of gold and platinum.

In the embodiment of FIG. 1, the at least one IR sensitive element comprises two IR sensitive elements 12 and 14, wherein the conductive electrode pads (22, 24, 26, 28, 30 and 32) and the two IR sensitive elements 12 and 14 are in a centerline symmetrical configuration in which the conductive electrode pads and the two IR sensitive elements are centerline symmetrical about the first axis 36 and the second axis 38, perpendicular to the first axis, in the plane of the infrared detector assembly 10. The centerline symmetrical configuration and a pad mid-portion of the thermistor conductive electrode pads that comprises a thermal heat loss reduction member of each thermistor conductive electrode pad (as discussed further herein below) are operable to advantageously reduce a thermal lag time between a temperature of the at least one thermistor 34 and a temperature of the at least one IR sensitive element (12, 14) during temperature transients of the infrared detector assembly 10 (e.g., of the IR sensitive element(s)). In particular, the centerline symmetrical configuration is preferably operable to reduce the thermal lag time between a temperature of the at least one thermistor and a temperature of the at least one IR sensitive element to one second or less during temperature transients of the infrared detector assembly.

Referring still to FIG. 1, forming of the conductive electrode pads (22, 24, 26, 28, 30 and 32) comprises depositing and patterning a conductive material overlying the substrate 16 into (i) at least one pair of first and second IR sensitive element conductive electrode pads (e.g., a first pair (22, 24) and a second pair (26, 28)) directly on the substrate 16 for each of the at least one IR sensitive element (12, 14) and (ii) first and second thermistor conductive electrode pads (indicated via reference numerals 30 and 32) directly on the substrate for each of the at least one thermistor 34 (yet to be formed or mounted). Depositing and patterning of the conductive material can be accomplished using suitable techniques known in the art.

Each pair of first and second IR sensitive element conductive electrode pads (e.g., first pair (22, 24) and second pair (26, 28)) electrically couple to a respective at least one IR sensitive element (e.g., first IR sensitive element 12 and second IR sensitive element 14, respectively). Electrical coupling is accomplished via an edge portion of the respective at least one IR sensitive element being overlapped by an edge portion of each respective pad of the pair of first and second IR sensitive element conductive electrode pads. This is shown in FIG. 1 with the use of phantom lines, as appropriate. In addition, the opposite end portions (i.e., opposite to the portion overlapping an IR sensitive element) of each pad of each pair of the first and second IR sensitive conductive electrode pads advantageously provide for a wire bond, or other suitable bond, that would provide electrical coupling of the respective IR sensitive element, via infrared signal leads (not shown), to external drive and measurement electronics (not shown), to be discussed further herein. In one embodiment, the first and second IR sensitive conductive electrode pads are in the shape of ovalized rectangles, i.e., a rectangle with rounded corners.

Still making reference to FIG. 1, each of the first and second thermistor conductive electrode pads, 30 and 32, respectively, has a plan view geometry of two pad end portions (indicated via reference numerals 40 and 42) spaced along a length dimension of a respective thermistor conductive electrode pad, the two pad end portions 40 and 42 having length and width dimensions and being joined via a pad mid-portion (indicated via reference numeral 44). The pad mid-portion 44 comprises a thermal heat loss reduction member having a width dimension less than its length dimension. The width dimension of the pad mid-portion 44 is less than the respective width dimension of each of the two pad end portions, 40 and 42. In one embodiment, each of the two pad end portions are in the shape of ovalized squares, i.e., a square with rounded corners.

The pad mid-portion 44 is thinner in width along its length than the two pad end portions 40 and 42 so as to advantageously reduce a potential for undesirable heat loss along the length of the respective conductive electrode pad, from one end thereof to the other. In addition, the combination of the dimensions of the pad mid-portion and the centerline symmetric features (as discussed herein) synergistically and advantageously minimize heat loss and reduce thermal gradients across the IR detector assembly. Any temperature gradients across the IR sensing elements and thermistor are kept consistent, i.e., uniform, with a thermal lag time of less than one second, to advantageously achieve a balanced thermal characteristic across the device. In addition, since the conductive electrode pads and the IR sensitive elements are disposed directly on the substrate 16, without any intervening layers, thermal lag is further advantageously minimized.

In one embodiment, the thermistor conductive electrode pads have a “barbell” type shape. The inventors discovered that a solely rectangular shape for the thermistor conductive electrode pads presents a problem with respect to undesirable heat loss, via the solely rectangular shape conductive electrode pad. Due to the small dimensions of the assembly components, a certain amount of substrate real estate is needed to electrically and thermally couple the thermistor thereto, but which also limits an amount of heat transmitted (e.g., heat loss) to an opposite pad end portion of each thermistor conductive pad (e.g., which serves as a bonding pad) where an electrical connection to a control or measurement circuit is to be made. In other words, the pad mid-portion 44 minimizes an amount of heat being undesirably pulled off the IR detector plate element, wherein the latter situation would create undesirable temperature differentials (ΔT's) between the IR detector elements and a remainder of the IR detector assembly.

Any place where there's a connection on the IR detector assembly that goes outside or external to the assembly, there exists a potential for heat loss, and a temperature differential across the part. If there's a temperature gradient across the assembly, then an undesirable difference in temperature between the PbSe IR sensitive elements of the first channel and second channel can occur. The embodiments of the present disclosure advantageously minimize undesirable temperature gradients across the assembly.

In addition, each of the first and second thermistor conductive electrode pads 30 and 32 extend in tandem along a line parallel to the length dimension of the at least one IR sensitive element, 12 or 14, immediately adjacent to the at least one IR sensitive element and separated there from by a thermal coupling separation spacing (i.e., indicated via reference numeral 45). In one embodiment, the thermal coupling separation spacing 45 is in a range of 0.10 to 0.30 mm. Such an arrangement places the thermistor or temperature sensing element 34 as near as possible to the at least one IR detector element, 12 and 14, without touching the at least one IR detector element. Because film layer resistance of the at least one IR detector element, 12 and 14, varies with temperature in addition to IR radiation, it is important that the temperature at the film layer is well known to a precise degree in order to compensate the IR measurement.

The method further comprises performing one selected from the group consisting of (i) forming the at least one thermistor 34 directly on the substrate 16 via a deposited resistive thermistor chemistry and dicing the substrate with (a) the conductive electrode pads, (b) the at least one IR sensitive element, and (c) the at least one thermistor into at least one individual infrared detector assembly 10 and (ii) dicing the substrate 16 with (a) the conductive electrode pads and (b) the at least one IR sensitive element into at least one individual partial infrared detector assembly (not shown), and completing the at least one individual partial infrared detector assembly (not shown) by disposing the at least one thermistor 34 directly on an individual diced substrate, via a surface mountable resistive thermistor chip.

In connection with forming the at least one thermistor 34 directly on the substrate 16 via the deposited resistive thermistor chemistry, each respective at least one thermistor 34 is (a) thermally coupled to the substrate 16 and (b) electrically coupled between opposite pad end portions (e.g., end portions 42) nearest one another of a respective pair of the first and second thermistor conductive electrode pads, 30 and 32. The opposite pad end portions (e.g., end portions 42) nearest one another of the respective pair of the first and second thermistor conductive electrode pads, 30 and 32, are spaced from one another by a thermistor element deposition placement distance (i.e., indicated via reference numeral 47) of the at least one thermistor 34. Techniques for depositing a resistive thermistor chemistry on a substrate are generally known in the art, and thus not described in further detail herein.

In connection with a secondary operation of dicing the substrate 16, having the conductive electrode pads and the at least one IR sensitive element already formed thereon, into at least one individual partial infrared detector assembly (not shown) and disposing the at least one thermistor 34 directly on an individual diced substrate, each respective at least one thermistor 34 is (a) thermally coupled to the individual diced substrate and (b) electrically coupled between opposite pad end portions (e.g., end portions 42) nearest one another of a respective pair of the first and second thermistor conductive electrode pads, 30 and 32. For example, the thermistor 34 can be bonded to the conductive electrode pads 30 and 32 via a silver filled epoxy, which creates both an electrical and thermally conductive bond. The opposite pad end portions (e.g., end portions 42) nearest one another of the respective pair of the first and second thermistor conductive electrode pads, 30 and 32, are spaced from one another by a surface mount thermistor placement distance (also indicated via reference numeral 47) of the at least one thermistor 34. In addition, the end portions 40 of the first and second thermistor conductive electrode pads, 30 and 32, advantageously provide for a wire bond, or other suitable bond, that would provide electrical coupling of the thermistor, via temperature signal leads (not shown), to external drive and measurement electronics (not shown), to be discussed further herein. Techniques for dicing a wafer and for surface mounting of components, such as a surface mount thermistor chip on a substrate, in addition to wire bonding or similar bonding technique, are generally known in the art, and thus not described in further detail herein.

According to additional embodiments of the present disclosure, the at least one IR sensitive element (12, 14) can comprise one selected from the group consisting of (i) a single IR sensitive element, (ii) two IR sensitive elements, and (iii) a plurality of IR sensitive elements. For instance, the at least one IR sensitive element can comprise two or more IR sensitive elements, wherein the conductive electrode pads and the two or more IR sensitive elements are in a centerline symmetrical configuration in which the conductive electrode pads and the two or more IR sensitive elements are centerline symmetrical about the first axis 36 and a second axis 38, perpendicular to the first axis, in the plane of the infrared detector assembly. In addition, in another embodiment, the at least one thermistor 34 can comprise two or more thermistors, wherein the conductive electrode pads and the at least one IR sensitive element are in a centerline symmetrical configuration in which the conductive electrode pads and the at least one IR sensitive element are centerline symmetrical about the first axis 36 and a second axis 38, perpendicular to the first axis, in the plane of the infrared detector assembly. In still another embodiment, the at least one IR sensitive element (12, 14) comprises one or more IR sensitive elements, wherein the at least one thermistor 34 comprises multiple thermistors, and wherein each of the at least one thermistors is disposed adjacent to at least one of the one or more IR sensitive elements.

The method herein advantageously provides for manufacturability of the IR detector assembly and further for an ability to obtain a device with more favorable thermal characteristics over prior art devices, such as thermal lag time in response to temperature transient events across the IR detector assembly. For example, the dicing of two IR sensitive elements at a time per IR detector assembly advantageously eliminates a need to carry out additional manufacturing steps of separate screening and/or matching of a pair of individual diced IR sensing elements for matched characteristics, e.g., thermal conductivity characteristics, Vₛₜₐᵣₜ operating characteristics, etc. In other words, the dicing of a pair of IR sensitive elements at a time, with mounting provisions for the addition of a thermistor closely coupled to the IR sensitive elements, provides benefits and advantages, e.g., with respect to matched characteristics, diced as a matched set, etc.

For example, in a system with two IR detector elements as shown in FIG. 1, one IR detector sensing element 12 is arranged to sample a gas, and the other IR detector sensing element 14 is used as a reference. Each IR detector sensing element includes its temperature being sensed via the at least one thermistor or temperature sensor 34.

In a further embodiment, both IR detector elements, 12 and 14, thermistor 34, and substrate 16 of FIG. 1, may additionally be mounted onto an optional means (not shown in FIG. 1) for heating and cooling the substrate 16. Such an optional heating and cooling means is illustrated and discussed herein below with respect a common heat spreader with reference to FIG. 6. By this later arrangement, the lead selenide film temperature of both detector elements can be very accurately measured and tracked. The lead selenide film temperature data can also be used (i) for temperature control of the additional optional means (see FIG. 6, heat spreader 152) for heating or cooling the substrate 16 by using the temperature measurement via the thermistor 34, and (ii) for detector temperature compensation algorithms to maintain accuracy of the overall gas sensing, e.g. capnography, system over a wide range of ambient temperatures.

The optional heating and cooling means may comprise any of a number of heating and cooling techniques, such as electrical Ni-Chrome heating filaments disposed underlying the substrate and driven by an external controller, Peltier cooling/heating, passive controlled heating using a resistive heater element with an integral metal heat spreading surface (e.g., a surface mount power resistor), a heater/cooler with an intermediate metal heat spreader (e.g. a metal heat sink) to allow heat flow to/from the detector substrate, and the like. Preferably, the temperature signal is provided via the temperature signal lead to a temperature control circuit (see FIG. 6, controller 210), which in turn determines a heating or cooling control input back to the heater/cooler means (FIG. 6, heat spreader 152), at a value sufficient to maintain the desired substrate temperature.

With reference now to FIG. 2, an electrical schematic of detection circuit 50 is shown for use with a dual channel detector assembly 10 having a single thermistor element 34 (Rₜₕₑᵣₘ) and two lead selenide detector elements 12 and 14 (Ch_1 R_{det} and Ch_2 R_{det}) according to an embodiment of the present disclosure.

The infrared detector assembly 10 of IR detector elements integrated with a temperature sensor (e.g., chip thermistor 34 and lead selenide IR sensitive elements 12, 14 thermally connected, as discussed) may be connected to an operating circuit 50 in a gas concentration measurement system. In particular, the circuit 50 of FIG. 2 is one embodiment of a circuit for generating a temperature compensated IR signal. One feature of circuit 50 is a common ground lead 52 shared by the temperature detector (Rₜₕₑᵣₘ ° C.) and each of the IR detector elements ground connections (Ch_1 R_{det} and Ch_2 R_{det}).

One of a bias voltage or current is passed through a resistive voltage divider to energize each of the lead selenide IR detector elements (Ch_1 R_{det} and Ch_2 R_{det}). In the FIG. 2 embodiment, the source is a voltage source 54. For the first channel, the resistive voltage divider is comprised of a bias resistor Ch_1 Rbias and IR detector resistance Ch_1 R_{det} through to ground 52, where Ch_1 Rbias is selected according to the desired output range. For the second channel, the resistive voltage divider is comprised of a bias resistor Ch_2 Rbias and IR detector resistance Ch_2 R_{det} through to ground 52, where Ch_2 Rbias is selected according to the desired output range. The resulting Ch_1 detector IR signal and Ch_2 detector IR signals, indicated via reference numerals 56 and 58, respectively, are output from the respective divider at the IR signal leads 60 and 62, respectively. IR signals 56 and 58 may be optionally amplified through detector amplifiers 64 and 66, or equivalent, to be further output as amplified IR detector signals 56a and 58a, respectively. The IR detector signals may then be further used by a gas detector system, described below, to provide system control functions, to be combined with other data for further signal processing, and/or to provide output information for displays and the like.

A separate bias voltage or current is applied through another resistive voltage divider to energize the temperature sensor or thermistor, Rtherm ° C. In the FIG. 2 embodiment, the source is a current source 68. The circuit arrangement allows the bias current through the thermistor Rtherm ° C. to be kept small, less than 50 μA. Small bias current helps to prevent self-heating of the thermistor, which if present would introduce undesirable temperature measurement errors.

The resistive voltage divider circuit for the temperature sensor 34 is comprised of another bias resistor Rbias and temperature sensor detector resistance Rtherm ° C. through to ground 52, where this Rbias is also selected according to the desired output range. The resulting temperature signal 70 is output from the divider at the temperature signal lead 72. Temperature signal 70 may be optionally amplified through a thermistor amplifier 74 or equivalent, to be further output as an amplified temperature signal 70a. The temperature detector signal may then be further used by a gas detector system, described below, to provide system control functions, to be combined with other data for further signal processing, and/or to provide output information for displays and the like. The temperature detector signal may also be used as a substrate temperature control signal in order to maintain the substrate and IR detectors at a desired temperature.

The arrangement described above enables further reductions in measurement errors. The actual lead selenide IR sensitive film temperature is advantageously measured to better than 0.01° C. accuracy by this arrangement. Accordingly, the better than 0.01° C. accuracy further advantageously allows for improved temperature compensation algorithms to be performed in downstream capnography measurements for carbon dioxide (CO₂) accuracy, i.e., over a broad range of ambient temperatures for which the capnography system can potentially be exposed to.

In addition, the embodiment of the IR detector assembly, as described herein with respect to FIGS. 1 and 2 and including dual channel lead selenide detector elements (e.g., sample and reference channels), advantageously provides for parallel output signals of temperature compensated IR signals. Such an arrangement is advantageous for gas detector assemblies which use both of a reference detector and a sample detector to simultaneously detect IR absorption characteristics of a gas stream at different frequencies. Many capnography systems use such an arrangement of reference detector and sample detector.

The approach to the lead selenide detector element and thermistor design, as described in the various embodiments herein, greatly improves an accuracy of measuring the lead selenide detector element temperature. In addition, the approach to lead selenide detector element and thermistor design of the present disclosure advantageously improves a response time for measuring small dynamic changes in temperature between the sample and reference channel lead selenide plate detectors. In particular, the centerline symmetrical configuration of the IR detector assembly advantageously reduces the thermal lag time between a temperature of the at least one thermistor and a temperature of the at least one IR sensitive element to one second or less during temperature transients of the infrared detector assembly.

With reference now to FIG. 3, there is shown an infrared detector assembly 10 with integrated temperature sensing according to another embodiment, including a top view and a side view of the key components of dual, surface mount, chip thermistors 34 and 80 integrated with dual lead selenide detector elements 12 and 14, for use as reference and sample channels, on a substrate 16. The embodiment of FIG. 3 is similar to that of the embodiment of FIG. 1 with the following differences. The at least one IR sensitive element comprises two IR sensitive elements 12 and 14. The conductive electrode pads (22, 24, 26, 28, 30, 32, 76 and 78) and the two IR sensitive elements 12 and 14 are in a centerline symmetrical configuration in which the conductive electrode pads and the two IR sensitive elements are centerline symmetrical about the first axis 36 and the second axis 38, perpendicular to the first axis, in the plane of the infrared detector assembly 10. In addition, the at least one thermistor comprises two thermistors, indicated via reference numerals 34 and 80. The conductive electrode pads, the two thermistors, and the at least one IR sensitive element are in a centerline symmetrical configuration in which the conductive electrode pads and the at least one IR sensitive element are centerline symmetrical about a first axis and a second axis, perpendicular to the first axis, in the plane of the infrared detector assembly. The embodiment of FIG. 3 advantageously provides for a dual channel IR detector assembly with dual thermistors, i.e., one thermistor per IR sensitive element.

With reference now to FIG. 4, there is shown an infrared detector assembly 10 with integrated temperature sensing according to another embodiment, including a top view of the key components of dual, surface mount, chip thermistors 34 and 80 integrated with a single lead selenide detector element 12, for use as a reference channel or a sample channel, on a substrate 16. The embodiment of FIG. 4 is similar to that of the embodiment of FIG. 1 with the following differences. The at least one IR sensitive element comprises one IR sensitive element 12. The conductive electrode pads (22, 24, 30, 32, 76 and 78) and the one IR sensitive element 12 are in a centerline symmetrical configuration in which the conductive electrode pads and the single IR sensitive element are centerline symmetrical about the first axis 36 and the second axis 38, perpendicular to the first axis, in the plane of the infrared detector assembly 10. In addition, the at least one thermistor comprises two thermistors, indicated via reference numerals 34 and 80. The conductive electrode pads, the two thermistors, and the at least one IR sensitive element are in a centerline symmetrical configuration in which the conductive electrode pads and the at least one IR sensitive element are centerline symmetrical about a first axis and a second axis, perpendicular to the first axis, in the plane of the infrared detector assembly. The embodiment of FIG. 4 advantageously provides for a single channel IR detector assembly with dual thermistors, i.e., one thermistor on each of two opposite sides of the single channel IR sensitive element.

Referring now to FIG. 5, there is shown an infrared detector assembly 10 with integrated temperature sensing according to another embodiment, including a top view of the key components of single, surface mount, chip thermistor 34 integrated with a single lead selenide detector element 12, for use as a reference channel or a sample channel, on a substrate 16. The embodiment of FIG. 5 is similar to that of the embodiment of FIG. 1 with the following differences. The at least one IR sensitive element comprises one IR sensitive element 12. The conductive electrode pads (22, 24, 30, and 32) and the one IR sensitive element 12 are in a centerline symmetrical configuration in which the conductive electrode pads and the single IR sensitive element are centerline symmetrical about the first axis 36 in the plane of the infrared detector assembly 10. In addition, the at least one thermistor comprises one thermistor, indicated via reference numeral 34. The conductive electrode pads, the single thermistor, and the at least one IR sensitive element are in a centerline symmetrical configuration in which the conductive electrode pads and the at least one IR sensitive element are centerline symmetrical about a first axis in the plane of the infrared detector assembly. The embodiment of FIG. 5 advantageously provides for a single channel IR detector assembly with a single thermistor, i.e., one thermistor on one side of the single channel IR sensitive element.

With reference now to FIG. 6, there is shown an improved capnography or carbon dioxide gas detector system 100 which includes an infrared detector assembly 10 having a dual IR detector with an integrated temperature sensor according to an embodiment of the present disclosure. The carbon dioxide gas detector system 100 integrates the improved IR detector assembly 10 in its assembly. The overall system, excluding improved detector 10, is somewhat similar to the assembly described in co-assigned U.S. Patent Publication No. 2013/0292570 entitled “System and method for performing heater-less lead selenide-based capnometry and/or capnography”, which is herein incorporated by reference. A sensor assembly 110 is configured to detect a level of carbon dioxide in a body of gas. The sensor assembly 110 employs the afore-described infrared detector assembly 10 with dual IR detector elements and integrated temperature sensor of FIG. 1. In the embodiment of FIG. 6, infrared radiation detector element 12 is arranged to capture an IR signal, and infrared radiation detector 14 is arranged to capture an IR reference signal. The detectors 12/14 may be lead selenide detectors. Measurements of sensor device 110 are compensated for variations in temperature at IR detector assembly 10 via temperature sensor or thermistor 34, as previously described. This may reduce the cost, enhance stability, enhance ruggedness, enhance manufacture and/or provide other advantages over prior sensor devices.

In one embodiment, sensor device 110 includes a “U” shaped housing 128 enclosing a source assembly 112, a hollow airway assembly 114, a detector assembly 116, and/or other components. Two opposing legs of the “U” shaped housing 128 define opposite sides of a gap there-between, with the source assembly 112 disposed in one leg on one side of the gap (source side) and the detector assembly 116 disposed in the opposing leg on the opposite side of the gap (detector side). The sensor device 110 may include self-contained electronics (not shown in FIG. 6) disposed within the housing 128.

The airway assembly 114 has windows 126 disposed on opposite sides such that infrared radiation entering the airway via the window 126 on one side of the airway 114 passes through a sample gas (patient respiration) in the airway 114 and exits via the window 126 on the opposite side. The airway assembly 114 may be either a disposable unit or a reusable unit that removably clips into the gap in the “U” shaped housing, with the source assembly 112 and detector assembly 116 being generally arranged such that infrared radiation emanating from the source assembly is directed across the gap through the gas sample in the airway assembly 114 to impinge upon the detector assembly 116. The airway windows 126 may be formed of plastic film (disposable version), sapphire (reusable version) and/or other materials.

The source assembly 112 includes a radiation source 118, optics 120, and/or other components. The emitter 118 may be driven by a pulsed source of energy to produce pulsed infrared radiation. The optics 120 may include a sapphire half-ball lens 122, a sapphire window 124, and/or other optical components. The radiation source 118 produces broadband radiation including an “MWIR” (Mid-Wavelength Infra-Red) band. Infrared radiation generally refers to radiation occupying a band of wavelengths in the optical spectrum between 0.7 μm and 300 μm. “MWIR” generally refers to a mid-wavelength subset of the infrared radiation band between 3 μm and 8 μm. MWIR radiation emitted by the radiation source 118 includes a reference wavelength and a carbon dioxide wavelength (λREF and λCO₂, respectively). The radiation source 118 may be pulsed at about 100 Hz to produce a periodically varying MWIR signal with a period of about 10 milliseconds. The sapphire half-ball lens 122 gathers and collimates the emitted radiation, directing it across the gap and through the airway assembly 114 towards the detector assembly 116 via the sapphire window 124.

The detector assembly 116 includes an optical system 130, an IR detector assembly 10 having dual IR detector elements (12 and 14) with integrated temperature sensor 34, and/or other components. The optical system 130 comprises a lens assembly 138, a beam splitter assembly 140, and/or other optical components. The lens assembly 138, which in one embodiment includes an AR-coated (Anti-Reflective coated) silicon plano-convex lens, focuses the MWIR radiation reaching it from the source assembly 112, and directs the electromagnetic radiation toward first IR radiation detector element 12 and second IR radiation detector 14 via beam splitter assembly 140. In beam splitter assembly 140, a dichroic beam-splitter 144 is positioned to reflect IR radiation containing the carbon dioxide wavelength λCO₂ towards first IR detector element 12, and to pass IR radiation containing the reference wavelength λREF towards second IR detector element 14 via a turning mirror 146. A narrow-band first optical filter 148 that passes λCO₂ is positioned in front of first IR detector element 12. A narrow-band second optical filter 150 that passes λREF is positioned in front of second IR detector element 14.

As previously described in connection with the discussion of FIG. 2, first and second IR detector elements 12, 14 are disposed on substrate 16, which may further be disposed on a common heat spreader 152. That is, the IR detector assembly 10 including the dual channel of two IR detector elements may optionally be mounted to a heat spreader 152. The IR detector assembly may be bonded onto the heat spreader of an actively heated or cooled substrate using a thermally conductive adhesive. The heat spreader 152 provides a means for heating and/or cooling the substrate 16, as discussed previously. In one embodiment, the heat spreader comprises a surface mount power resistor to provide only heating to some temperature above ambient. Power for heating in this embodiment is supplied via a temperature control input 250 from an external power supply under control of a temperature control or controller circuit 210. A control input to the control circuit 210 may be received from the electrical temperature signal lead 234. An output from control circuit 210 for controlling the temperature of the substrate 16 is then used to drive the heating power input at temperature control input 250. A temperature control loop results.

In an embodiment of the IR detector assembly that comprises more than one thermistor, e.g., two thermistors or dual thermistors, then either of the two thermistors can be used to measure detector temperature and also be used as the feedback variable term for the temperature control loop, e.g., to maintain a desired constant substrate temperature. Alternatively, an average temperature from both chip thermistors could be used for the feedback temperature value in the temperature control loop.

Even with the temperature compensated IR detection assembly's detector elements 12 and 14 (e.g., chip thermistor and lead selenide detector elements) mounted to a common substrate 16, the substrate will experience some degree of differential temperature gradients across the heat spreader 152, e.g., substrate heater or cooler. These temperature gradients may be accounted for and can be algorithmically compensated for in real-time in the measurement controller circuit to maintain overall capnometer system accuracy over a wide ambient operating range. The mounting of the chip thermistor as close as possible to the IR detector film deposition layer enables the film temperature to be measured to better than 0.01° C. accuracy for each of the two detectors. Any mismatch or drift in temperature between the two detectors is accurately measured by this arrangement, enabling the downstream processing and control circuits to apply very accurate temperature corrections to the IR signal.

With reference still to FIG. 6, IR signal outputs from IR signal leads 212, 214 provide a desired IR detection signal and an IR reference signal, respectively to a gas detector controller circuit 210. A temperature signal output from temperature signal lead 234 provides a temperature signal from the IR detectors (e.g., sensed via the thermistor 34) to controller circuit 210, as well. Controller 210 processes signals 212, 214 and 234 to obtain a temperature-compensated IR signal and a corresponding temperature-compensated carbon dioxide gas concentration value from the sample gas crossed by the optical path. Controller 210 further outputs the carbon dioxide value to an output 220, which may be a visual display.

Controller 210 may optionally provide a temperature control output that is a function of temperature signal 234 in order to maintain the temperature of the IR detector assembly 10 at a desired value. The temperature control output is experienced by IR detector assembly 10 as input 230 to temperature control input 250 of heat spreader 152. The temperature control algorithm may be any of that described herein, equivalents, or as known in the art.

Also in accordance with the principles of the present invention, a method is described which incorporates the above summarized apparatus to measure a lead selenide plate detector temperature, and which results in an improved and quicker temperature measurement. The embodiments of the present disclosure advantageously enable a full thermal stabilization across the IR detector assembly, from ambient (or room temperature) to 50° C. within 15 to 60 seconds, and more preferably under 30 seconds. Such a characteristic can be referred to as the device thermal stabilization characteristic with respect to thermal lag as heat goes across the IR detector assembly. Minimal thermal lag in the system is desired so that temperature changes can be detected quickly, thereby providing for improved accuracy of IR detection capabilities.

Turning now to FIG. 7, a flow chart is shown illustrating a method 300 for measuring a gas concentration according to another embodiment of the present disclosure. In connection with the method, the controller 210 of FIG. 6 preferably includes circuitry (as shown in FIG. 2) to provide the functions and steps involved in the method 300 for measuring a gas concentration. The method 300 begins with a first step 302 of providing an infrared detector assembly having IR radiation detector elements with integrated temperature sensor as described previously and in relation to FIG. 1 and/or FIGS. 3-5. Controller 210 then provides a step 304 of inputting a constant current source to one or more of the temperature sensors via the temperature signal lead(s), and a step 306 of inputting a voltage source to one or more of the IR detector element film layers via the IR detector signal lead(s). IR detector assembly 10 with dual channel IR detector elements responsively provides outputs of temperature and IR signals from leads (234) and (212, 214), respectively. Controller 210 receives the outputs in the obtaining step 308 of obtaining a temperature signal from the temperature signal lead and the receiving step 310 of receiving an IR signal from the IR detector signal lead. The obtaining step 308 may further include amplifying the temperature signal, and the receiving step 310 may further include amplifying the IR detector signals, both amplifying by means of amplifying circuits (74) and (64, 66), respectively (see FIG. 2).

Controller 210 further executes a step 312 of compensating for a drift of the IR signal from the receiving step 310 based on the obtaining step 308. Controller 210 then provides the step 316 of outputting a measurement of a gas concentration, preferably to output 220 (FIG. 6), based upon the compensating and receiving steps.

Controller 210 optionally executes a step 314 of controlling the temperature of the substrate based upon the temperature signal from the obtaining step 308. As previously described, controller 210 may use one or more temperature inputs 70 (FIG. 2), 234 (FIG. 6) to provide a control signal 230 to temperature control input 250 of the common heat spreader 152, wherein control signal 230 controls the heating or cooling energy of the heat spreader 152 to maintain the substrate 16 at a controlled and desired temperature.

One benefit of being able to control the temperature of substrate 16 (in addition to the positioning of the at least one thermistor as close as possible to the lead selenide detector elements, and the centre line symmetry, as discussed herein) is advantageously being able to obtain a more accurate representation of the temperature of the PbSe for active temperature compensation. This further advantageously enables the IR detector assembly to be operated out of range for the detector element. In other words, the device can be actively heated to a specific set temperature, wherein beyond that specific set temperature, the device goes out of regulation, i.e., the system can drift. By accurately monitoring the temperature of the IR detector elements, via the thermistor and conductive pads as discussed herein, the IR detector assembly can be operated outside its regulated temperature, to an elevated operating temperature range, e.g., to advantageously obtain an additional 15° C. in operating range, beyond its regulated temperature.

Although only a few exemplary embodiments have been described in detail above, those skilled in the art will readily appreciate that many modifications are possible in the exemplary embodiments without materially departing from the novel teachings and advantages of the embodiments of the present disclosure. For example, the embodiments of the present disclosure, and various configurations of thermistors for temperature detection which fulfill the objectives of the described embodiments, can be advantageously used in capnography, gas spectroscopy, lead selenide detectors, mid-range infrared spectroscopy, e.g., with respect to measuring exhaled patient gasses. Accordingly, all such modifications are intended to be included within the scope of the embodiments of the present disclosure as defined in the following claims. In the claims, means-plus-function clauses are intended to cover the structures described herein as performing the recited function and not only structural equivalents, but also equivalent structures.

In addition, any reference signs placed in parentheses in one or more claims shall not be construed as limiting the claims. The word “comprising” and “comprises,” and the like, does not exclude the presence of elements or steps other than those listed in any claim or the specification as a whole. The singular reference of an element does not exclude the plural references of such elements and vice-versa. One or more of the embodiments may be implemented by means of hardware comprising several distinct elements, and/or by means of a suitably programmed computer. In a device claim enumerating several means, several of these means may be embodied by one and the same item of hardware. The mere fact that certain measures are recited in mutually different dependent claims does not indicate that a combination of these measures cannot be used to an advantage.

1. A method of making an infrared detector assembly with integrated temperature sensing, the method comprising:
forming at least one infrared radiation sensitive element or IR sensitive element on a substrate, wherein the at least one IR sensitive element is thermally coupled to the substrate;
forming conductive electrode pads for (a) the at least one IR sensitive element and (b) at least one thermistor on the substrate, wherein the conductive electrode pads are thermally coupled to the substrate, and wherein the conductive electrode pads and the at least one IR sensitive element are in a centerline symmetrical configuration in which the conductive electrode pads and the at least one IR sensitive element, taken together, are centerline symmetrical about at least one axis in a plane of the infrared detector assembly, wherein the centerline symmetrical configuration is operable to reduce a thermal lag time between a temperature of the at least one thermistor and a temperature of the at least one IR sensitive element during temperature transients of the infrared detector assembly, wherein forming the conductive electrode pads comprises depositing and patterning a conductive material overlying the substrate into
(i) at least one pair of first and second IR sensitive element conductive electrode pads on the substrate for each of the at least one IR sensitive element, wherein each pair of first and second IR sensitive element conductive electrode pads electrically couple to a respective at least one IR sensitive element via an edge portion of the respective at least one IR sensitive element overlapped by an edge portion of each respective pad of the pair of first and second IR sensitive element conductive electrode pads, and
(ii) first and second thermistor conductive electrode pads on the substrate for each of the at least one thermistor, wherein each of the first and second thermistor conductive electrode pads has a plan view geometry of two pad end portions spaced along a length dimension of a respective thermistor conductive electrode pad, the two pad end portions having length and width dimensions and being joined via a pad mid-portion, wherein the pad mid-portion comprises a thermal loss reduction member having a width dimension less than its length dimension, further wherein the width dimension of the pad mid-portion is less than the respective width dimension of each of the two pad end portions, further wherein each of the first and second thermistor conductive electrode pads extend in tandem along a line parallel to the length dimension of the at least one IR sensitive element, immediately adjacent to the at least one IR sensitive element and separated there from by a thermal coupling separation spacing; and
performing one selected from the group consisting of
(i) forming the at least one thermistor on the substrate via a deposited resistive thermistor chemistry, wherein each respective at least one thermistor is (a) thermally coupled to the substrate and (b) electrically coupled between opposite pad end portions nearest one another of a respective pair of the first and second thermistor conductive electrode pads, wherein the opposite pad end portions nearest one another of the respective pair of the first and second thermistor conductive electrode pads are spaced from one another by a thermistor element deposition placement distance of the at least one thermistor, and dicing the substrate with the conductive electrode pads, the at least one IR sensitive element, and the at least one thermistor (34) into at least one individual infrared detector assembly, and
(ii) dicing the substrate with the conductive electrode pads and the at least one IR sensitive element into at least one individual partial infrared detector assembly, and completing the at least one individual partial infrared detector assembly by disposing the at least one thermistor on an individual diced substrate, via a surface mountable resistive thermistor chip, wherein each respective at least one thermistor is (a) thermally coupled to the individual diced substrate and (b) electrically coupled between opposite pad end portions nearest one another of a respective pair of the first and second thermistor conductive electrode pads, wherein the opposite pad end portions nearest one another of the respective pair of the first and second thermistor conductive electrode pads are spaced from one another by a surface mount thermistor placement distance of the at least one thermistor.

2. The method according to claim 1, wherein the substrate comprises a quartz substrate having a thickness in a range of 0.50 to 0.70 mm, and wherein the at least one IR sensitive element comprises a lead selenide film element.

3. The method according to claim 1, wherein the thermal coupling separation spacing is in a range of 0.10 to 0.30 mm.

4. The method according to claim 1, wherein (i) the at least one pair of first and second IR sensitive element conductive electrode pads and (ii) the first and second thermistor conductive electrode pads of the at least one thermistor comprise a single electrically conductive material or more than one electrically conductive material, wherein each of the more that one electrically conductive materials is of an at least 90-100% matched thermal conductivity.

5. The method according to claim 1, wherein the centerline symmetrical configuration of the conductive electrode pads and the at least one IR sensitive element is operable to reduce the thermal lag time between a temperature of the at least one thermistor and a temperature of the at least one IR sensitive element to one second or less during temperature transients of the infrared detector assembly.

6. The method according to claim 1, wherein the conductive electrode pads comprise at least one of gold and platinum.

7. The method according to claim 1, wherein the at least one IR sensitive element comprises one selected from the group consisting of (i) a single IR sensitive element, (ii) two IR sensitive elements, and (iii) a plurality of IR sensitive elements.

8. The method according to claim 1, further wherein the at least one IR sensitive element comprises two or more IR sensitive elements, and wherein the conductive electrode pads and the two or more IR sensitive elements are in a centerline symmetrical configuration in which the conductive electrode pads and the two or more IR sensitive elements are centerline symmetrical about a first axis and a second axis, perpendicular to the first axis, in the plane of the infrared detector assembly.

9. The method according to claim 1, further wherein the at least one thermistor comprises two or more thermistors, and wherein the conductive electrode pads and the at least one IR sensitive element are in a centerline symmetrical configuration in which the conductive electrode pads and the at least one IR sensitive element are centerline symmetrical about a first axis and a second axis, perpendicular to the first axis, in the plane of the infrared detector assembly.

10. The method according to claim 1, further wherein the at least one IR sensitive element comprises one or more IR sensitive elements,
wherein the at least one thermistor comprises multiple thermistors, and
wherein each of the at least one thermistors is disposed adjacent to at least one of the one or more IR sensitive elements.

11. An infrared detector assembly with integrated temperature sensing, comprising:
at least one infrared radiation sensitive element or IR sensitive element formed on a substrate, wherein the at least one IR sensitive element is thermally coupled to the substrate;
conductive electrode pads formed on the substrate for (a) the at least one IR sensitive element and (b) at least one thermistor, wherein the conductive electrode pads are thermally coupled to the substrate, and wherein the conductive electrode pads and the at least one IR sensitive element are in a centerline symmetrical configuration in which the conductive electrode pads and the at least one IR sensitive element, taken together, are centerline symmetrical about at least one axis in a plane of the infrared detector assembly, wherein the centerline symmetrical configuration is operable to reduce a thermal lag time between a temperature of the at least one thermistor and a temperature of the at least one IR sensitive element during temperature transients of the infrared detector assembly, wherein the conductive electrode pads comprise
(i) at least one pair of first and second IR sensitive element conductive electrode pads on the substrate for each of the at least one IR sensitive element, wherein each pair of first and second IR sensitive element conductive electrode pads electrically couple to a respective at least one IR sensitive element via an edge portion of the respective at least one IR sensitive element overlapped by an edge portion of each respective pad of the pair of first and second IR sensitive element conductive electrode pads, and
(ii) first and second thermistor conductive electrode pads on the substrate for each of the at least one thermistor, wherein each of the first and second thermistor conductive electrode pads has a plan view geometry of two pad end portions spaced along a length dimension of a respective thermistor conductive electrode pad, the two pad end portions having length and width dimensions and being joined via a pad mid-portion, wherein the pad mid-portion comprises a thermal loss reduction member having a width dimension less than its length dimension, further wherein the width dimension of the pad mid-portion is less than the respective width dimension of each of the two pad end portions, further wherein each of the first and second thermistor conductive electrode pads extend in tandem along a line parallel to the length dimension of the at least one IR sensitive element, immediately adjacent to the at least one IR sensitive element and separated there from by a thermal coupling separation spacing; and
at least one thermistor selected from the group consisting of
(i) at least one thermistor formed on the substrate via a deposited resistive thermistor chemistry, wherein each respective at least one thermistor is (a) thermally coupled to the substrate and (b) electrically coupled between opposite pad end portions nearest one another of a respective pair of the first and second thermistor conductive electrode pads, wherein the opposite pad end portions nearest one another of the respective pair of the first and second thermistor conductive electrode pads are spaced from one another by a thermistor element deposition placement distance of the at least one thermistor, and
(ii) at least one thermistor mounted on the substrate that comprises a surface mountable resistive thermistor chip, wherein each respective at least one thermistor is (a) thermally coupled to the substrate and (b) electrically coupled between opposite pad end portions nearest one another of a respective pair of the first and second thermistor conductive electrode pads, wherein the opposite pad end portions nearest one another of the respective pair of the first and second thermistor conductive electrode pads are spaced from one another by a surface mount thermistor placement distance of the at least one thermistor.

12. The infrared detector assembly according to claim 11, wherein the substrate comprises a quartz substrate having a thickness in a range of 0.50 to 0.70 mm,
wherein the at least one IR sensitive element comprises a lead selenide film element,
wherein the thermal coupling separation spacing is in a range of 0.10 to 0.30 mm,
wherein (i) the at least one pair of first and second IR sensitive element conductive electrode pads and (ii) the first and second thermistor conductive electrode pads of the at least one thermistor comprise a single electrically conductive material or more than one electrically conductive material, wherein each of the more that one electrically conductive materials is of an at least 90-100% matched thermal conductivity, and
wherein the centerline symmetrical configuration of the conductive electrode pads and the at least one IR sensitive element is operable to reduce the thermal lag time between a temperature of the at least one thermistor and a temperature of the at least one IR sensitive element to one second or less during temperature transients of the infrared detector assembly.

13. The infrared detector assembly according to claim 11, wherein the conductive electrode pads comprise at least one of gold and platinum.

14. The infrared detector assembly according to claim 11, wherein the at least one IR sensitive element comprises one selected from the group consisting of (i) a single IR sensitive element, (ii) two IR sensitive elements, and (iii) a plurality of IR sensitive elements.

15. The infrared detector assembly according to claim 11, further wherein the at least one IR sensitive element comprises two or more IR sensitive elements, and wherein the conductive electrode pads and the two or more IR sensitive elements are in a centerline symmetrical configuration in which the conductive electrode pads and the two or more IR sensitive elements are centerline symmetrical about a first axis and a second axis, perpendicular to the first axis, in the plane of the infrared detector assembly.

16. The infrared detector assembly according to claim 11, further wherein the at least one thermistor comprises two or more thermistors, and wherein the conductive electrode pads and the at least one IR sensitive element are in a centerline symmetrical configuration in which the conductive electrode pads and the at least one IR sensitive element are centerline symmetrical about a first axis and a second axis, perpendicular to the first axis, in the plane of the infrared detector assembly.

17. The infrared detector assembly according to claim 11, further wherein the at least one IR sensitive element comprises one or more IR sensitive elements,
wherein the at least one thermistor comprises multiple thermistors, and
wherein each of the at least one thermistors is disposed adjacent to at least one of the one or more IR sensitive elements.

18. A carbon dioxide gas measurement apparatus comprising the infrared detector assembly according to claim 11, wherein the carbon dioxide gas measurement apparatus further comprises:
a circuit coupled to the infrared detector assembly and configured to (i) obtain a temperature measurement output from the at least one thermistor and (ii) provide a temperature compensated carbon dioxide gas measurement output signal based on the obtained temperature measurement, wherein the circuit compensates an output signal of the at least one IR sensitive element for a drift in temperature of the respective at least one IR sensitive element in response to the obtained temperature measurement.

19. The carbon dioxide gas measurement apparatus of claim 18, wherein the at least one IR sensitive element comprises two IR sensitive elements, and wherein the conductive electrode pads and the two IR sensitive elements are in a centerline symmetrical configuration in which the conductive electrode pads and the two IR sensitive elements are centerline symmetrical about a first axis and a second axis, perpendicular to the first axis, in the plane of the infrared detector assembly, and
wherein one of the two IR sensitive elements is configured to output an IR reference signal, and the other of two IR sensitive elements is configured to output a carbon dioxide gas measurement signal, wherein both the reference signal and the carbon dioxide gas measurement signal are temperature compensated for a drift in temperature of each respective IR sensitive element in response to the obtained temperature measurement.

20. A method of measuring a gas concentration comprising:
providing an infrared detector assembly according to claim 11;
obtaining, via a circuit coupled to the infrared detector assembly, a temperature measurement output from the at least one thermistor; and
providing, via the circuit, a temperature compensated carbon dioxide gas measurement output signal based on the obtained temperature measurement, wherein an output signal of the at least one IR sensitive element is compensated, via the circuit, for a drift in temperature of the respective at least one IR sensitive element in response to the obtained temperature measurement.
